(19) United States
(12) Patent Application Publication
(10) Pub. No.: US 2004/0158247 A1
(43) Pub. Date: Aug. 12, 2004

(54) Polyaxial pedicle screw system

(76) Inventors: Arthit Sitiso, Arleta, CA (US); John Wagner, Chatsworth, CA (US); Frank Bailly, San Pedro, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/773,135

(22) Filed: Feb. 4, 2004

Related U.S. Application Data
(60) Provisional application No. 60/445,524, filed on Feb. 7, 2003.

(51) Int. Cl. A61B 17/56

(52) U.S. Cl. 606/61

(57) ABSTRACT

A spinal fixation system for use in the mobilization of a sequence of spinal bones includes a unique pedicle screw assembly. The pedicle screw assembly includes a pedicle screw having a head portion pivotally secured within a base of a body and a threaded shaft portion extending therefrom for insertion into the spinal bone. The body includes a rod passageway adapted to receive a rod extending between two or more pedicle screw assemblies. The body includes oppositely threaded internal and external threads. A set screw having exterior threads engages the internal threads of the body so as to be selectively disposed therein and in contact with the rod. A nut having internal threads engages the external threads of the body. The set screw and nut are preferably simultaneously fastened by turning in opposite directions to counteract the forces applied to the assembly and spinal bones.

### RELATED APPLICATION

This application claims priority to U.S. Provisional Patent Application Serial No. 60/445,524 filed Feb. 7, 2003.

### BACKGROUND OF THE INVENTION

The present invention generally relates to spinal fixation systems, such as those used in the treatment of degenerative spinal diseases. More particularly, the present invention relates to a spinal fixation system having an improved polyaxial pedicle screw assembly which utilizes counter rotation tightening to ensure stabilization of the vertebrae during the installation of the system.

The human spine is a complex columnar structure of vertebral bone and connective tissues. The vertebrae, disk and ligaments are intricately arranged and complex interaction amongst these structures provides flexibility for motion, spinal cord protection and distribution of body forces. In the diseased or injured state, this delicate equilibrium is disturbed and results in spinal pathologies. In many cases, the spinal disorders can be treated by conservative non-surgical methods, such as medication, exercise or physical therapy. However, some spinal disorders, such as degenerative instability, deformity, trauma, and tumors require surgical intervention to treat pain induced by nerve root compression and unstable invertebral joints.

The surgical procedures for the spine involve the dissection of soft tissues and often the removal of load bearing structures, such as vertebral bone and disks to decompress the neural elements. Such decompression procedures lead to spinal instability and it is often necessary to fuse spinal segments to restore the stability.

Pedicle screw fixation systems, and other spinal fixation systems, are well-known and used when the spine and vertebrae are damaged or degenerating, such as in the cases of some cancers, and the like. Such spinal fixation systems are intended to provide immobilization and stabilization of spinal segments in skeletally-mature patients as an adjunct to fusion in the treatment of various acute and chronic instabilities or deformities of the thoracic lumbar and sacral spine such as degenerative spondylolisthesis, fractured vertebrae, dislocation, scoliosis, kyphosis, spinal tumor, or previously failed fusions. Typically, such fixation systems are installed in the vertebrae of skeletally-mature patients receiving fusion by autogenous bone graft having implants attached to the lumbar and sacral spine, with removal of the implants after the attainment of a solid fusion.

A variety of internal spinal fixation systems have been developed and used in spine surgery to achieve such a rigid fixation by implanting artificial assemblies in or on the spine. Such spinal implants can generally be classified as anterior or posterior instrumentation systems based upon the implanting location. Anterior implants are coupled to the anterior portion of the spine. The use of posterior implants using pedicle screws coupled by longitudinal rods has become more popular because of their capability of achieving a rigid fixation. Such posterior implants generally comprise pairs of rods which are aligned along the longitudinal axis of the spine, and which are then attached to the spinal column by screws which are inserted through the pedicles into respective verebral bodies.

The surgical procedure to achieve a posterior fixation using pedicle screws and connecting rods generally includes the insertion of screws into pedicles in a predetermined angle and depth, temporary coupling of the rods to the screws, the proper correction of spinal curve, and the secure connection of rods to the screws for a rigid fixation.

A considerable difficulty identified in the surgical procedure is associated with the coupling of a rod to a plurality of screws that are not well aligned in general because the angle and depth of the screw insertion should be determined by a patients anatomical and pathological conditions that may vary among spinal levels as well as among patients. It has been identified that attempts for addressing such a difficulty result in the application of unnecessarily excessive loads to the spinal column near the pedicle screws and the increased operation time, which are known to cause many complications associated with surgery.

The use of polyaxial screws, however, has been used in coupling the screw with the rod as the pedicle screw and coupling element assembly provide a polyaxial freedom of implantation angulation with respect to rod reception. An example of such a polyaxial pedicle screw is disclosed in U.S. Pat. No. 5,961,518. However, in this patent, the screw and rod assembly are locked using a nut oriented sideways (i.e. the tightening nut faces the lateral side of the patient during surgery), which is very inconvenient to perform in a very limited lateral operating space. In fact, most surgeons prefer the top-tightening mechanism (i.e. the axis of tightening member faces the posterior side of the patient) because the top-tightening mechanism provides better visibility and access than the side-tightening mechanism.

Another example of such a spinal fixation system is offered by the Forex Corporation under the Global Spinal Fixation System trade name. This system is the subject of U.S. Pat. No. 6,280,443, the contents of which are hereby incorporated by reference. This spinal fixation system is an internal fixation device for spinal surgery which comprises pedicle screws, connectors, rods, screw caps, hooks and transverse link assemblies. Although performing generally adequate, the Forex system is very complicated and can require three or four hours of surgery to properly install.

Also, in such spinal fixation systems, when tightening the various components, the vertebrae are often twisted due to the large amounts of pressure applied to the nuts, set screws, pedicle screws, etc. Thus, the amount of torque or pressure applied to such systems is limited, or damage can be done to the vertebrae or even the assembly due to the twisting and turning of the vertebrae during the tensioning process.

Accordingly, there is a continuing need for a more simple spinal fixation assembly which stabilizes the vertebrae during the installation of the system and allows a greater torque or pressure to be applied to the components thereof to ensure a tight and stable securement to the vertebrae. The present invention fulfills this need and provides other related advantages.

### SUMMARY OF THE INVENTION

The present invention resides in a spinal fixation system incorporating a pedicle screw assembly which allows the implantation of the pedicle screws at the best anatomic location and orientation with flexibility of screw placement and alignment; easy and simple connection between the rod and the screw assembly; and a top-tightening mechanism wherein counter-active torque forces are applied during tightening of the assembly to ensure maximum tightening while eliminating harmful twisting forces on the vertebrae.

The spinal fixation system of the present invention generally comprises a plurality of pedicle screw assemblies and a rod extending between pedicle screw assemblies between vertebrae. Each pedicle screw assembly includes a pedicle screw, a body, a set screw and a nut. The body has an aperture adapted for receiving the threaded portion of the screw therethrough and retaining at least a portion of the head in a base of the body. The body also includes a rod passageway adapted to receive the rod therein, and oppositely threaded internal and external threads.

The head portion of the pedicle screw is preferably rounded such that the head and base of the body form a spherical joint such that the body and pedicle screw can pivot and rotate with respect to one another. Typically, a compression washer is disposed in the base, such as press-fitting the compression washer therein, for retaining the head of the pedicle screw within the base. The compression washer preferably includes a concave facet disposed above the head of the pedicle screw to facilitate pivoting of the pedicle screw and body.

The pedicle screw includes a drive slot formed in the head portion thereof for insertion into the vertebrae. In a particularly preferred embodiment, the threaded portion of the pedicle screw is tapered. That is, although a major diameter of the threaded portion is generally constant in diameter, a minor diameter of the threaded portion is tapered to prevent the pedicle screw from becoming loose or exiting the vertebrae over time.

The set screw has exterior threads for engaging the internal threads of the body. The set screw includes a drive slot therein and is adapted to travel within the body and contact the rod, securing it in place within the body.

The nut has internal threads for engaging the external threads of the body. Typically, the nut has a polygonal outer configuration for tightening by a socket device or the like. The invention preferably uses a tightening tool for simultaneously tightening the set screw and the nut such that the set screw and nut are fastened in opposite directions simultaneously to counteract fastening forces applied to the assembly. Such tightening device may comprise a wrench having a handle, a shaft extending therefrom and a socket at the end of the shaft which is adapted to engage the nut. A driver has a handle at an end of the shaft which is slidably extended through the shaft of the wrench. A driver end of the driver engages the driver slot of the set screw. The handles of the driver and wrench can be turned in opposite directions to simultaneously tighten both the set screw and the nut.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a perspective view of an assembled polyaxial pedicle screw assembly embodying the present invention;

FIG. 2 is an exploded perspective view of the assembly of FIG. 1, illustrating the component parts thereof;

FIG. 3 is a top view of a spinal fixation system incorporating the pedicle screw assembly of the present invention for securing adjacent spinal bones, in phantom, to one another;

FIG. 4 is a cross-sectional view of the pedicle screw assembly taken generally along line 4-4 of FIG. 1;

FIG. 5 is a cross-sectional view similar to FIG. 4, illustrating the pivotal connection between a screw and body of the assembly;

FIG. 6 is a partially fragmented and sectioned view of a tightening device used in accordance with the present invention engaging a set screw and nut of the assembly of the present invention; and

FIG. 7 is a partially fragmented and sectioned view similar to FIG. 6, illustrating the opposite rotation of the set screw and nut to counteract torque forces in accordance with the present invention when tightening the assembly.

### DETAILED DESCRIPTION OF THE PREFERRED EMBODIMENT

As shown in the drawings for purposes of illustration, the present invention resides in a spinal fixation system 10 which is used in spinal degenerative diseases and deformities to provide immobilization and stabilization of spinal segments as an adjunct to fusion, such as bone graft implants and the like.

FIG. 3 illustrates two pair of polyaxial pedicle screw assemblies 12 inserted into adjoining vertebrae 14 and interconnected by rods 16. Typically, the system 10 is installed in the spinal segment of skeletally-mature patients which are receiving fusion by autogenous bone grafts having implants which are attached to the spine, with subsequent removal of the implants after the attainment of a solid fusion-usually within eight to ten months or less.

Although FIG. 3 illustrates two vertebrae which have been immobilized and stabilized by two pair of interconnected polyaxial pedicle screw assemblies 12, it will be appreciated by those skilled in the art that three, four or even more vertebrae may be immobilized and stabilized with the use of additional pedicle screw assemblies 12 and longer rods or additional rods. In some instances, cross-linking members may be used to interconnect the generally parallel rods 16, depending upon the need of each case and the desire of the surgeon. However, the system 10 of the present invention generally does not require such cross-links.

It will also be noted that FIG. 3 illustrates the rods 16 as being generally straight. In prior systems, the rods were required to be bent in order to interconnect the pedicle screws or provide the proper lardosis. However, in the present invention, the rods 16 typically do not need to have a bend, saving valuable surgical time. The rods 16 of the present invention may be bent as desired by the surgeon or in certain complicated cases wherein several vertebrae are being fused together and immobilized by the system 10 of the present invention.

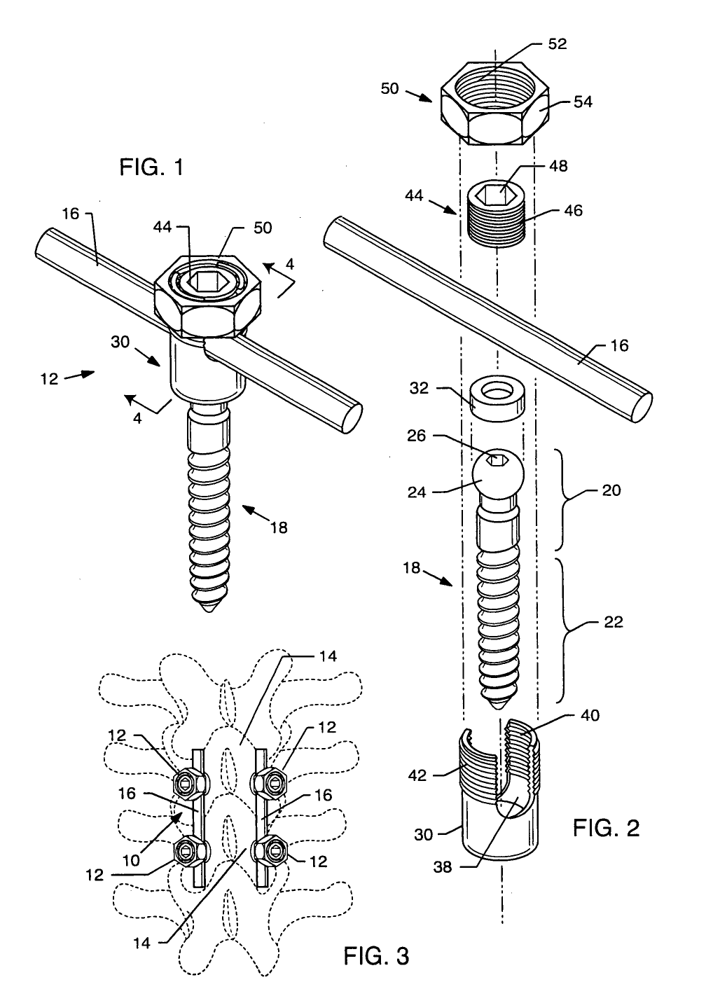

With reference to FIGS. 1-4, typically, all components of the system 10 are comprised of a strong and biocompatible material, such as titanium or a titanium-based alloy, such as Ti6Al4V ELI. The assembly 12 is comprised of a pedicle screw 18 having an upper head portion 20 and a lower threaded portion 22 extending downwardly therefrom. As shown in FIG. 2, the head portion 20 includes a rounded or spherical head 24. A drive slot 26 is formed in the head 24, typically a hexdepression for insertion of an Allen wrench or similar driver in order to drive the screw 18 into the vertebrae or other spinal bone 14, as shown in FIG.3 and as is well-known in the art. The threaded portion of the pedicle screw 22 extends through an aperture 28 of a base portion of a body 30.

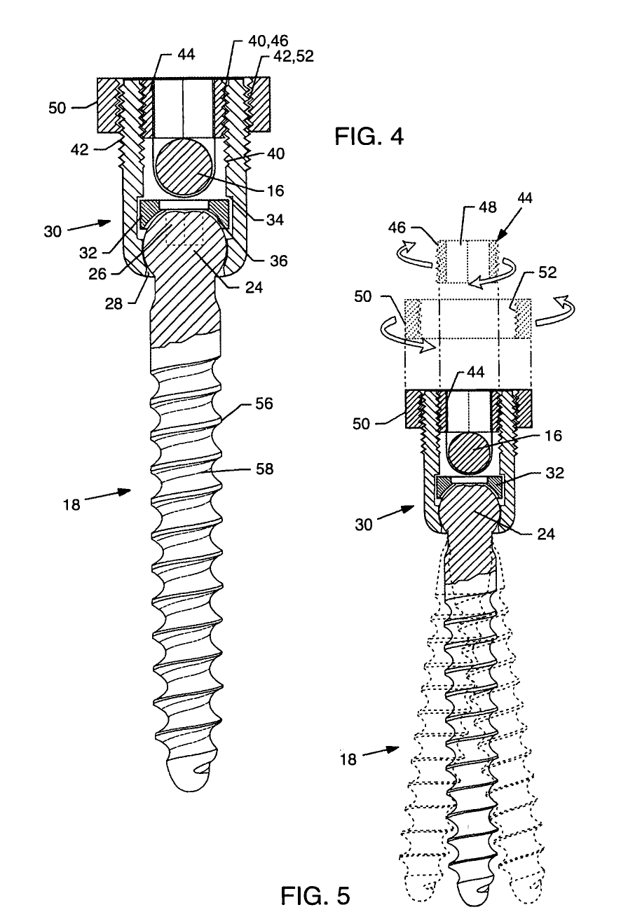

The aperture 28 is configured such so as to not prevent the spherical head 24 from extending therethrough. Instead, the aperture is generally defined by a concave rim, as illustrated in FIG. 4. In a particularly preferred embodiment, a compression washer 32 is press fit within the base portion of the body above the generally spherical head of the pedicle screw 18. The compression washer 32 resides within a base of the body 30 beneath a ledge 34 thereof. This prevents the screw 18 from being removed from the body 30. In fact, to the surgeon, the body 30, compression screw 32 and pedicle screw 18 appear as a single unit due to their interconnection. The washer 32, in a particularly preferred embodiment, has a generally concave facet 36 such that a spherical joint is created between the head 24 of the pedicle screw 18, the body 30 and the washer 32. This enables the body 30 and pedicle screw 18 to pivot and rotate with respect to one another. Typically, such degree of pivoting is approximately 15°. This enables the pedicle screw 18 to be inserted into the bone and the body 30 to pivot somewhat for reception of a rod, as illustrated in FIG. 5.

With particular reference to FIGS. 2 and 4, the body 30 includes a rod passageway, illustrated as an open-ended generally U-shaped slot 38. FIG. 1 illustrates the rod 16 extending through the rod passageway 38 of the body 30. An upper portion of the body 30 includes internal threads 40 and external threads 42. It is of importance to the present invention that these threads 40 and 42 be oppositely threaded. As illustrated, the internal threads 40 are right hand threads while the external threads 42 are left hand threads. The right or left thread can be altered so long as the internal and external threads 40 and 42 are oppositely threaded.

A set screw 44 is sized and configured such so as to be received within the upper open end of the body 30. The set screw 44 has external threads 46 which are adapted to engage the internal threads 40 of the body 30. As illustrated, the set screw 44 has external right hand threads which engage the internal right hand threads 40 of the body 30. The set screw 44 has a drive slot 48 formed therein for selective placement within the body 30. The drive slot 48 is typically of an internal hexagon configuration for engagement with a hexagon Allen or driver.

A nut 50 is sized and configured so as to be disposed over a top portion of the body 30. The nut 50 includes internal threads 52 which are adapted to engage the external threads 42 of the body 30. As illustrated, the internal threads 52 of the nut 50 would be left handed so as to engage the left handed threads 42 of the body 30. Preferably, the nut 50 is of a polygonal, and typically hexagonal, exterior circumferential conformation so as to be received within a socket wrench or the like, as will be described more fully herein.

In use, the pedicle entry point is prepared in the spinal bone structure, as is well-known in the art. An appropriate driver is then inserted into drive socket 26 such that the pedicle screw 18 is fastened within the bone structure, with the head portion 20 and body 30 remaining above the bone, as illustrated in FIG. 3. The same process is repeated for the adjacent spinal bone vertebrae.

Some prior art systems in the past have used straight pedicle screws. However, it has been found that such screws can become loosened over time and self-removed, at least partially, from the spinal bone structure. In order to eliminate this possibility, the present invention utilizes a tapered thread portion of the pedicle screw 18. That is, the major outer diameter 56 of the threaded portion is generally constant in diameter. However, the inner minor diameter 58 of the pedicle screw 18 is increasingly tapered from the head portion 20 to the tip of the screw 18. This creates a thread taper which serves to securely lock the pedicle screw 18 in place within the spinal bone.

With reference again to FIG. 3, once the pedicle screw assemblies 12 are fastened in place in adjoining vertebrae, a rod 16 is extended through the rod passageways 38 of adjoining bodies 30, which extend above the vertebral bone. Due to the pivoting nature of the body 30 with respect to the screw 18, the body 30 can be pivoted with respect to one another until properly aligned for insertion of the rod 16 therethrough. The set screw 44 is then inserted into the body 30 until it contacts the rod 16, as illustrated in FIG. 4. The nut 50 is then threaded onto the exterior of the body 30. Initially, the set screw 44 and nut 50 are somewhat loosely fastened onto the body 30. For final tightening, the set screw 44 and nut 50 are simultaneously turned to tighten the assembly. This simultaneous opposite turning serves to counteract the torque forces experienced by the assembly 12 and the connected vertebral bones 14. This allows the assembly 12 to be tightened to a great degree without placing undue strain on the assembly 12 or the underlying vertebral bones 14. The end result is that the rod 16 is securely tightened between two now interconnected pedicle screw assemblies 12, as illustrated in FIG. 3.

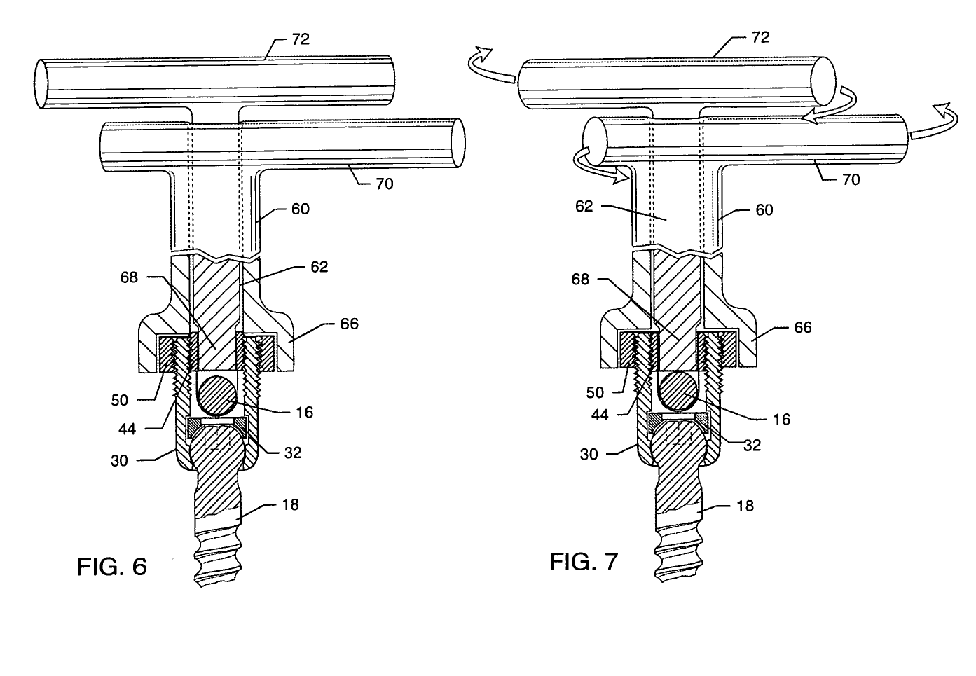

In a particularly preferred embodiment, as illustrated in FIGS. 6 and 7, a tool which is adapted to tighten the system 10 of the present invention is used. The tool includes a socket wrench 60 having an internal conduit or passageway 62 for reception of a driver 64 therethrough. The lower end of the socket wrench 60 defines a hex socket 66, which is placed over the hex nut 50 so as to engage and tighten the hex nut 50. A lower end of the driver 64 defines an Allen wrench point 68 which is inserted into the internal hexagon socket 48 of the set screw 44 in order to tighten it.

A handle 70 of the socket wrench 60 is turned in a counter-clockwise, or left-hand turn to tighten the nut 50 while the handle 72 of the driver 64 is turned in a clockwise or right-hand turn to simultaneously tighten the set screw 44. By using both hands to turn the handles 70 and 72 in opposite directions in order to tighten the nut 50 and set screw 44, the assembly 12 can be tightened to a great degree while ensuring stabilization of the vertebrae during the counter-rotation and tightening of the assembly 12. Such counter-rotation also provides ease of installation and ensures proper alignment of the body 30 to correct alignment of interconnecting rod 16 while maintaining proper alignment of the vertebrae. Additionally, whereas 80 pounds of torque or pressure, for example, is utilized in prior art systems to tighten these systems, utilizing the counter-rotation assembly of the present invention allows a doubling of this pressure or torque to ensure a firm and tight connection, as well as proper alignment and stabilization of the vertebrae. Additionally, the simple design of the system 10 of the present invention enables a surgeon to install the system in much less time than prior systems, typically less than two hours.

Although several embodiments have been described in detail for purposes of illustration, various modifications may be made without departing from the scope and spirit of the invention. Accordingly, the invention is not to be limited, except as by the appended claims.

What is claimed is:

1. A pedicle screw assembly for use in a spinal fixation system, the assembly comprising:
a pedicle screw having a head portion and a threaded shaft portion extending therefrom;
a body having an aperture adapted for receiving the threaded portion of the screw therethrough while retaining at least a portion of the head portion in a base of the body, a rod passageway, and oppositely threaded internal and external threads;
a set screw having exterior threads for engaging the internal threads of the body; and
a nut having internal threads for engaging the external threads of the body.

2. The assembly of claim 1, wherein the head portion of the pedicle screw includes a rounded head, and wherein the head and base form a spherical joint such that the body and head pivot with respect to one another.

3. The assembly of claim 2, including a compression washer disposed in the base for retaining the head of the pedicle screw within the base.

4. The assembly of claim 3, wherein the compression washer is press-fit within the base and includes a concave facet disposed above the head of the pedicle screw.

5. The assembly of claim 1, wherein the threaded portion of the pedicle screw is tapered.

6. The assembly of claim 5, wherein a major diameter of the threaded portion is generally constant, and wherein a minor diameter of the threaded portion is tapered.

7. The assembly of claim 1, wherein the pedicel screw includes a drive slot formed in the head portion thereof.

8. The assembly of claim 1, wherein the rod passageway and the pedicle screw aperture of the body are generally transverse to one another.

9. The assembly of claim 1, including a rod extending through the rod passageway.

10. The assembly of claim 9, wherein the set screw is adapted to travel within the body and contact the rod, securing it in place within the body.

11. The assembly of claim 1, wherein the set screw includes a drive slot therein for tightening by a driver device.

12. The assembly of claim 1, wherein the nut has a polygonal outer configuration for tightening by a socket device.

13. A polyaxial pedicle screw assembly for use in a spinal fixation system, the assembly comprising:
a pedicle screw including a head portion having a rounded head and a drive slot therein and a threaded shaft portion extending therefrom;
a body having an aperture adapted for receiving the threaded portion of the screw therethrough while retaining the rounded head in a base of the body, a rod passageway generally transverse to the pedicle screw aperture, and oppositely threaded internal and external threads;
a rod extending through the rod passageway;
a set screw having exterior threads for engaging the internal threads of the body and having a drive slot for selectively being moved into contact with the rod to secure the rod within the body;
a nut having internal threads for engaging the external threads of the body;
wherein the round head of the screw and the base form a spherical joint permitting pivoting therebetween; and
wherein the set screw and nut are fastened in opposite directions to counteract fastening forces applied to the assembly.

14. The assembly of claim 13 including a compression washer disposed in the base for retaining the head of the pedicle screw within the base.

15. The assembly of claim 14, wherein the compression washer is press-fit within the base and includes a concave facet disposed above the head of the pedicle screw.

16. The assembly of claim 13, wherein the threaded portion of the pedicle screw is tapered.

17. The assembly of claim 16, wherein a major diameter of the threaded portion is generally constant, and wherein a minor diameter of the threaded portion is tapered.

18. A spinal fixation system, comprising:
a plurality of pedicle screw assemblies; and
a rod extending between the pedicle screw assemblies;
wherein each pedicle screw assembly comprises:
a pedicle screw including a head portion having a rounded head and a threaded shaft portion extending therefrom;
a body having an aperture adapted for receiving the threaded portion of the screw therethrough while retaining the rounded head in a base of the body to permit the screw and body to pivot with respect to one another, a rod passageway adapted for insertion of the rod therethrough, and oppositely threaded internal and external threads;
a set screw having exterior threads for engaging the internal threads of the body and having a drive slot for being selectively inserted into the body and in contact with the rod to secure the rod in place within the body; and
a nut having internal threads for engaging the external threads of the body.

19. The system of claim 18, including a compression washer disposed in the base above the screw for retaining the head of the screw within the base.

20. The system of claim 18, wherein a major diameter of the threaded portion is generally constant, and wherein a minor diameter of the threaded portion is tapered.

21. The system of claim 18, wherein the pedicle screw includes a drive slot formed in the head portion thereof.

22. The system of claim 18, including a tightening device for simultaneously tightening the set screw and the nut.

23. The system of claim 22, wherein the tightening device comprises a wrench having a handle, a shaft and a socket adapted to engage the nut, and a driver having a handle, a shaft slidably extending through the shaft of the wrench and a driver end for engaging the driver slot of the set screw.
